(19) United States
(12) Patent Application Publication
(10) Pub. No.: US 2025/0316354 A1
(43) Pub. Date: Oct. 9, 2025

(54) Systems and Methods for Voice Activation and Annotation of Medical Records

(71) Applicant: Physio-Control, Inc., Redmond, WA (US)

(72) Inventors: Ryan Apperson, Redmond, WA (US); Tyson Heo, Redmond, WA (US); Michelle Liu, Redmond, WA (US); Michelle Robershotte, Redmond, WA (US); Mark Rutzer, Redmond, WA (US); Tyson Taylor, Redmond, WA (US); Dennis Sohn, Redmond, WA (US)

(21) Appl. No.: 19/169,278

(22) Filed: Apr. 3, 2025

Related U.S. Application Data
(60) Provisional application No. 63/573,905, filed on Apr. 3, 2024.

(51) Int. Cl. G16H 15/00; G10L 15/26; G16H 10/60

(52) U.S. Cl. CPC G16H 15/00; G10L 15/26; G16H 10/60

(57) ABSTRACT

Example implementations relate to annotating a patient electronic health record. An example method includes detecting an annotation trigger based on user input. In response to detecting the annotation trigger, the method can include obtaining a first audio data segment containing speech uttered by a user. The example method can further includes determining that the first audio data segment containing speech includes at least one marker word that is associated with at least one marker event in a pre-defined set of marker events. The example method can include determining a marker time at which the annotation trigger was detected, or at which the first audio data segment containing speech was uttered, and annotating the patient electronic health record with the marker event at a time point representing the marker time. The method can also include adding the annotated patient electronic health record to a master medical record.

### CROSS-REFERENCE TO RELATED APPLICATION

The present application claims the benefit of U.S. Provisional Patent Application No. 63/573,905, filed Apr. 3, 2024. The contents of which are hereby incorporated by reference in their entirety.

### BACKGROUND

Unless otherwise indicated herein, the description in this section is not prior art to the claims in this application and is not admitted to be prior art by inclusion in this section.

In emergency treatment situations, a healthcare provider may need to activate features of a physiological signal monitor. For example, the healthcare provider may need to ready the physiological signal monitor to deliver a shock. However, the healthcare provider's hands may be tied up in other activities related to the emergency treatment. Additionally, access to the physiological signal monitor or the buttons and/or screen of the physiological signal monitor might be limited due to the constraints of the emergency situations.

Furthermore, during medical events healthcare providers must document treatment of patients. The documentation can be used for post-medical event report generation. An example post-medical event report is an airway report, which requires annotation of the time of endotracheal tube placement, the time of paralytic administration, and the time of transfer to hospital care. The post-medical event reports must include accurate annotations of the treatments. These reports can be used to further diagnose and treat patients and can also be used for determining how the healthcare provider could have improved treatment during the medical event.

Various approaches have been developed to address some of the problems or circumstances related to activating physiological signal monitor actions and annotating medical records gathered by physiological signal monitors. However, the prior approaches suffer from problems or limitations of their own. Some approaches for annotating medical records include using drop down menus to select a medical event, or manually annotating the medical records after the event. These approaches still require great amounts of effort and can include inaccuracies. An additional approach for activating physiological signal monitor actions can include adding designated buttons on the physiological signal monitor. However, a healthcare provider would still need to use a free hand press the designated button.

### SUMMARY

Some implementations of the present disclosure generally relate to devices, systems, and methods for voice activating physiological signal monitor actions and annotations on medical records. The present disclosure may use wake word processing to trigger the annotation or medical records or the activation of a physiological signal monitor feature.

As such, in one aspect a method is provided for annotating a patient electronic health record. The method includes detecting an annotation trigger based on user input. In response to detecting the annotation trigger, the method further includes obtaining a first audio data segment containing speech uttered by a user, and determining that the first audio data segment containing speech includes at least one marker word that is associated with at least one marker event in a pre-defined set of marker events. The method further includes determining a marker time at which the annotation trigger was detected, or at which the first audio data segment containing speech was uttered, and annotating the patient electronic health record with the marker event that is associated with the at least one marker word. In an example implementation, the marker event is annotated within the patient electronic health record at a time point representing the marker time. The method further includes adding the annotated patient electronic health record to a master medical record.

In another aspect, a system is provided. The system includes a physiological signal monitor configured to monitor a patient, a microphone device configured to obtain speech from a user associated with the physiological signal monitor, and a controller. The controller includes at least one processor, at least one non-transitory data storage and a non-transitory computer-readable medium that stores a set of program instructions and a speech recognition system. The at least one processor executes the program instructions stored in the at least one non-transitory data storage and executable by the at least one processor to carry out a plurality of operations.

The operations include listening to speech uttered by a user, and detecting an action trigger present in the speech uttered by the user. In response to detecting the action trigger, the operations include obtaining an audio data segment containing speech from the user. The operations further include determining that the audio data segment containing speech from the user includes a physiological signal monitor command. After determining that the physiological signal monitor command is valid, and the operations include instructing the physiological signal monitor to perform a physiological signal monitor action associated with the physiological signal monitor command.

In another aspect, a method for physiological signal monitor control is provided. The method includes listening, with a microphone, to speech uttered by a user, and detecting a trigger word present in the speech uttered by the user. In response to detecting the trigger word, the method further includes obtaining an audio data segment including speech from the user. The method also includes determining that the audio data segment including speech from the user includes an activation. The activation includes at least one of a command or annotation. The method further includes sending instructions to a physiological signal monitor based on the activation.

These as well as other aspects, advantages, and alternatives will become apparent to those of ordinary skill in the art by reading the following detailed description, with reference, where appropriate, to the accompanying drawings.

### BRIEF DESCRIPTION OF THE DRAWINGS

FIG. 1 is a simplified block diagram of an example system in which various described principles can be implemented.

FIG. 2 is a simplified block diagram of an example physiological signal monitor.

FIG. 3 is a simplified block diagram of an example computing system in which various described principles can be implemented.

FIG. 4 is a diagram illustrating a representation of an example scene showing the use of a defibrillator for monitoring and delivering treatment to a person or patient experiencing a medical condition.

FIG. 5 is a front view of a defibrillator.

FIG. 6 illustrates a simplified block diagram of the components of a defibrillator.

FIG. 7 is a flow chart of an example method.

FIG. 8A illustrates a GUI of an external defibrillator displaying a patient treatment event list.

FIG. 8B illustrates a GUI of an external defibrillator displaying waveforms for a patient treatment event list.

FIG. 9 is a flow chart of an additional example method.

### DETAILED DESCRIPTION

The figures and the following description illustrate specific example methods, systems, and/or non-transitory computer readable mediums. It will be appreciated that those skilled in the art will be able to devise various arrangements that, although not explicitly described or shown herein, embody the principles described herein and are included within the scope of the claims that follow this description. Furthermore, any examples described herein are intended to aid in understanding the principles of the disclosure and are to be construed as being without limitation. As a result, this disclosure is not limited to the examples described below, but by the claims and their equivalents.

Particular example methods, systems, and/or non-transitory computer readable mediums are described herein with reference to the drawings. In the description, common features may be designated by common reference numbers throughout the drawings. In some drawings, multiple instances of a particular type of feature may be used. Although these features are physically and/or logically distinct, the same reference number is used for each, and the different instances are distinguished by addition of a letter to the reference number. When the features as a group or a type are referred to herein (e.g., when no particular one of the features is being referenced), the reference number is used without a distinguishing letter. However, when one particular feature of multiple features of the same type is referred to herein, the reference number is used with the distinguishing letter.

As used herein, various terminology is used for the purpose of describing particular implementations only and is not intended to be limiting. For example, the singular forms “a,” “an,” and “the” are intended to include the plural forms as well, unless the context clearly indicates otherwise. Further, the terms “comprise,” “comprises,” and “comprising” are used interchangeably with “include,” “includes,” or “including.” Additionally, the term “wherein” is used interchangeably with the term “where.” As used herein, “exemplary” indicates an example, an implementation, and/or an aspect, and should not be construed as limiting or as indicating a preference or a preferred implementation. As used herein, an ordinal term (e.g., “first,” “second,” “third,” etc.) used to modify an element, such as a structure, a component, an operation, etc., does not by itself indicate any priority or order of the element with respect to another element, but rather merely distinguishes the element from another element having a same name (but for use of the ordinal term). As used herein, the term “set” refers to a grouping of one or more elements, and the term “plurality” refers to multiple elements.

### I. Overview

As previously mentioned, in emergency treatment situations, a healthcare provider may need to activate features on a physiological signal monitor, but may not have the ability to press any buttons on the physiological signal monitor. For example, the healthcare provider may be performing chest compressions for CPR, but may also want to ready the physiological signal monitor (e.g. a defibrillator) to deliver a shock. In emergency situations it could be imperative to access or activate the physiological signal monitor without hands. Additionally, access to the physiological signal monitor or the buttons and/or screen of the physiological signal monitor might be limited due to the constraints of the emergency situation. For example, the emergency could be taking place in a tight room, corridor, or other crowded scene, which limits accessibility. In a further example, during patient transport the provider may want to start a noninvasive blood pressure measurement, or transmit a care record or other data to a hospital without unbuckling from their seat.

Further problems existing in terms of data gathering and annotations in the medical field include accurately documenting treatment of patients for post-medical event report generation. For example, a response team may be short-staffed, which may result in challenges in accurately documenting time stamps for post-medical event reports. An example post-medical event report is an airway report, which requires annotation of the time of endotracheal tube placement, the time of paralytic administration, and the time of transfer to hospital care. The post-medical event reports must include accurate annotations of the treatments, including accurate documentation of the time at which the treatments were administered. The reports can be used to further diagnose and treat patients and can also be used for determining how the healthcare provider could have improved treatment during the medical event.

Current attempt to address, and possibly ease, these issues have included adding designated buttons to the physiological signal monitor in order to cut down on the amount of interaction needed. However, the designated buttons still require the healthcare provider to stop what they are doing in order to press the button. Further, previous attempts to utilize speech recognition was unsuccessful due to inaccuracies. In emergency situations, the physiological signal monitor incorrectly interpreting the command could be catastrophic.

Additional approaches have been developed to address some of the problems related to activating physiological signal monitor actions and annotations on medical records gathered by physiological signal monitors. For example, one approach for annotating medical records includes using drop down menus to select a medical event. Although this may reduce the amount of annotation time required, it still takes additional time and can take attention away from the patient. Another approach includes manually annotating the medical records after the event. However, this approach still requires great amounts of time and effort and can include inaccuracies in the annotations, especially with extended time between the event and the act of annotating.

Examples provided herein describe a system for performing hands free actions with a physiological signal monitor configured to monitor a patient. For example, a defibrillator. The physiological signal monitor could be operated by a user (e.g., a healthcare provider) for emergency treatment. The system further includes a microphone device to obtain speech from a user associated with the physiological signal monitor. The microphone device may be incorporated within the physiological signal monitor, or could be part of a separate device. The system may include a controller with at least one processor, at least one non-transitory data storage and a non-transitory computer-readable medium that stores a set of program instructions and a speech recognition system. The at least one processor executes the program instructions to carry out operations.

The operations include listening to speech uttered by the user. For example, the microphone device previously mentioned can “listen to” the user's speech. Based on listening to speech uttered by the user via the microphone, the operations also include detecting an action trigger present in the speech uttered by the user. The action trigger could be a short command verbally given by the user for the physiological signal monitor to listen for a further call to action. For example, the command may be to listen for notes for the device to annotate into a patient record. In response to detecting the action trigger, the operations include obtaining an audio data segment comprising speech from the user. The operations further include determining that the audio data segment comprising speech from the user includes a physiological signal monitor command. For example, the command could be to start charging the defibrillator, or to add an annotation to the patient record. In order to confirm that the command was interpreted accurately, the operations further include determining if the physiological signal monitor command is valid, for example by comparing the command to a pre-determined list. Finally, the operations include instructing the physiological signal monitor to perform a physiological signal monitor action associated with the physiological signal monitor command.

Examples provided herein are further directed toward a method for physiological signal monitor control. The method can include listening, with a microphone, to speech uttered by a user. The user could be a healthcare provider, as previously mentioned. The method can further include detecting a trigger word present in the speech uttered by the user. The trigger word could be similar to a “wake word” in that it triggers the microphone to listen for a specific term. In response to detecting the trigger word, the method includes obtaining an audio data segment comprising speech from the user. The method further includes determining that the audio data segment comprising speech from the user includes an activation. In an example implementation, the activation includes at least one of a command or annotation. For example, the command could be to start charging for shock, and the annotation could be that the patient refused treatment. The method further includes sending instructions to a physiological signal monitor based on the activation.

Implementations provided herein also include a method for annotating a patient electronic health record. The example includes detecting an annotation trigger based on user input. For example, the user input could be tapping a button on a physiological signal monitor or uttering a wake word. Once the annotation trigger is detected, the implementation includes obtaining a first audio data segment containing speech uttered by a user. The audio data segment may be obtained using a microphone that is part of the physiological signal monitor with the button, previously mentioned, or could be on a different device. In response to detecting the annotation trigger, the implementation may include obtaining, with the microphone, a first audio data segment containing speech uttered by a user. The audio data segment may include one marker word, that is associated with at least one marker event in a pre-defined list of marker events. For example, the list could include “patient refused treatment,” “patient was nonresponsive,” or “administered a paralytic.” A marker time at which the annotation trigger was detected, or at which the first audio data segment containing speech was uttered, could be associated with the marker event. The annotated patient electronic health record is then added to a master medical record.

### II. Example Architecture

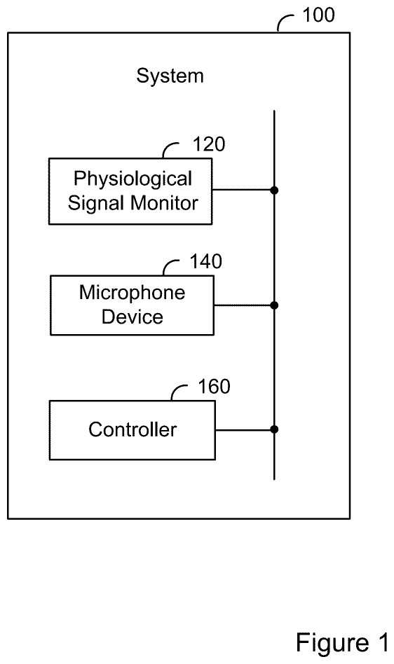

FIG. 1 is a simplified block diagram of an example system 100 in which various described principles can be implemented. This is accomplished using a physiological signal monitor 120, a microphone device 140, and a controller 160. The physiological signal monitor 120, the microphone device 140, and the controller 160 are all in communication with each other, but need not be part of the same device. Alternatively, the physiological signal monitor 120, microphone device 140, and controller 160 could all be part of the same device. For example, the physiological signal monitor could include the microphone device 140 and the controller 160.

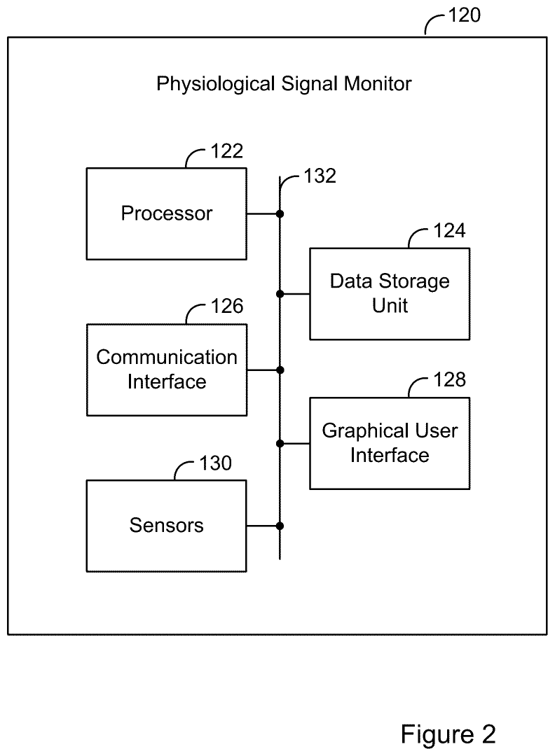

FIG. 2 is a simplified block diagram of an example physiological signal monitor 120. The physiological signal monitor 120 can be configured to perform and/or can perform various operations, such as the operations described in this disclosure. The physiological signal monitor 120 can include various components such as a processor 122, a data storage unit 124, a communication interface 126, a graphical user interface 128, sensors 130, and/or electrical connectors 132. Electrical connectors 132 can be represented by lines 132 that connect components of the physiological signal monitor 120, as shown in FIG. 2.

The processor 122 can be or can include a general-purpose processor (e.g., a microprocessor) and/or a special-purpose processor (e.g., a digital signal processor). The processor 122 can execute program instructions included in the data storage unit 124 as described below.

The data storage unit 124 can be or can include one or more volatile, non-volatile, removable, and/or non-removable storage components, such as magnetic, optical, and/or flash storage, and/or can be integrated in whole or in part with the processor 122. Further, the data storage unit 124 can be or can include a non-transitory computer-readable storage medium, having stored thereon program instructions (e.g., compiled or non-compiled program logic and/or machine code) that, upon execution by the processor 122, cause the physiological signal monitor 120 and/or another computing system to perform one or more operations, such as the operations described in this disclosure. These program instructions can define, and/or be part of, a discrete software application.

In some instances, the physiological signal monitor 120 can execute program instructions in response to receiving an input, such as an input received via the communication interface 126 and/or the graphical user interface 128. The data storage unit 124 can also store other data, such as any of the data described in this disclosure.

The communication interface 126 can allow the physiological signal monitor 120 to connect with and/or communicate with another entity according to one or more protocols. Therefore, the physiological signal monitor 120 can transmit data to, and/or receive data from, one or more other entities according to one or more protocols. In one example, the communication interface 126 can be or include a wired interface, such as an Ethernet interface or a High-Definition Multimedia Interface (HDMI). In another example, the communication interface 126 can be or include a wireless interface, such as a cellular or WI-Fi interface.

The graphical user interface 128 can allow for interaction between the physiological signal monitor 120 and a user of the physiological signal monitor 120. For example, the user can send instructions and receive feedback via the graphical user interface 128. As such, the graphical user interface 128 can be or include an input component such as a keyboard, a mouse, a remote controller, a microphone, and/or a touch sensitive panel. The graphical user interface 128 can also be or include an output component such as a display screen (which, for example, can be combined with a touch sensitive panel) and/or a sound speaker.

The sensors 130 can gather physiological information about the patient. The sensors 130 play a role in monitoring, diagnosing, and treating patients by converting various physiological and environmental parameters into measurable electrical signals. The sensors 130 are designed to capture information such as heart rate, blood pressure, body temperature, blood oxygen levels (pulse oximetry), respiratory rate, glucose levels, and more. They are utilized in a wide range of physiological signal monitors and systems, including patient monitors, wearable health trackers, medical imaging equipment, infusion pumps, ventilators, and diagnostic devices.

The sensors 130 can be many different kinds of sensors. For example, the sensors 130 can be electrocardiogram sensors, pulse oximeters, blood pressure sensors, temperature sensors, respiratory rate sensors, glucose sensors, infrared thermometers, flow sensors, pressure sensors, imaging sensors, accelerometers, pH sensors, gas sensors, electrodes, impedance sensors and/or motion sensors.

The physiological signal monitor 120 can also include one or more connection mechanisms that connect various components within the physiological signal monitor 120. For example, the physiological signal monitor 120 can include the connection mechanisms represented by lines of the electrical connectors 132 that connect components of the physiological signal monitor 120, as shown in FIG. 2.

The elements in the physiological signal monitor 120 may be electrically connected by the electrical connectors 132. The electrical connectors 132 electrically connect the processor 122, the data storage unit 124, the communication interface 126, the graphical user interface 128, and the sensors 130. The electrical connectors 132 may facilitate the current flowing through the physiological signal monitor. They may also facilitate the transmission of power, signals, and data for the functioning of the physiological signal monitor. Alternatively, the elements of the physiological signal monitor could be wirelessly connected to each other. For example, the sensors 130 could be in wireless communication with the rest of the elements. Alternatively, the data storage unit 124 could be stored remotely and in wireless communication. Alternatively still, the graphical user interface 128 could be in wireless communication with the other elements.

The physiological signal monitor 120 can include one or more of the above-described components and can be configured or arranged in various ways. For example, the physiological signal monitor 120 can be configured as a server and/or a client (or perhaps a cluster of servers and/or a cluster of clients) operating in one or more server-client type arrangements, such as a partially or fully cloud-based arrangement, for instance.

In some cases, the physiological signal monitor 120 can take the form of a different and/or more specific type of computing system, such as a desktop or workstation computer, a laptop, a tablet, a television, a set-top box, a media player, and/or a head-mountable display device (e.g., virtual-reality headset or an augmented-reality headset), among numerous other possibilities.

Further, as illustrated in FIG. 1, in one implementation, the system 100 could include the microphone device 140 configured to obtain speech from a user associated with the physiological signal monitor. Microphone device 140 could be a condenser microphone, a dynamic microphone, or an electret microphone. Microphone device 140 could also be capable of cancelling noise through noise cancellation or noise reduction. For example, microphone device 140 may suppress unwanted background noise while preserving desired audio signals. This can be achieved using techniques such as passive noise isolation, where physical barriers or materials are employed to attenuate external sounds, and active noise cancellation, where electronic circuitry within the microphone device 140 detects and analyzes incoming audio signals, identifying noise components to be canceled out. In active noise cancellation, an anti-noise signal is generated to counteract the noise, effectively reducing its presence in the final audio output. This process enhances the clarity and intelligibility of desired audio signals, making them more discernible amidst noisy environments or backgrounds, thus improving the overall quality of audio recordings or communications.

In one implementation, microphone device 140 could be a near field microphone. The near field microphone can capture audio signals in close proximity to the sound source, typically within a few inches or centimeters. Near-field microphones are tailored to excel at capturing detailed, high-fidelity audio from sources positioned nearby. They achieve this by utilizing specialized designs, such as small diaphragms or pressure-gradient configurations, which allow for precise localization and accurate reproduction of sound waves. Near-field microphones work by directly capturing the pressure variations produced by the sound source in close proximity, without significant contributions from distant reflections or environmental noise. This enables them to capture subtle nuances, transient details, and spatial characteristics of the sound with exceptional clarity and accuracy.

Alternatively, microphone device 140 could be a far field microphone. The far field microphone is a type of microphone designed to capture audio signals from sources positioned at a distance, typically several feet or meters away. Far-field microphones are engineered to effectively pick up sound from afar while minimizing the influence of ambient noise and reflections. They achieve this by utilizing techniques such as beamforming, which involves combining signals from multiple microphone elements to create directional sensitivity patterns that focus on desired sound sources while rejecting unwanted noise from other directions. Far-field microphones work by detecting pressure variations in the air caused by sound waves, converting them into electrical signals that can be processed and analyzed.

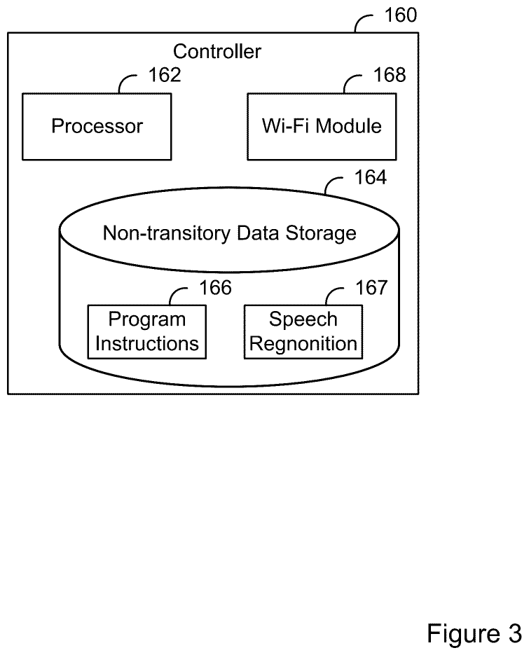

As further illustrated in FIG. 3, the system 100 could include a controller 160. The controller 160 includes at least one processor 162, at least one analog to digital converter, and a memory. The memory may include a computer readable medium. The computer readable medium may be a non-transitory data storage 164, which may include, without limitation, read-only memory (ROM), programmable read-only memory (PROM), erasable programmable read-only memory (EPROM), electrically erasable programmable read-only memory (EEPROM), non-volatile random-access memory (e.g., flash memory), a solid state drive (SSD), a hard disk drive (HDD), a Compact Disc (CD), a Digital Video Disk (DVD), a digital tape, read/write (R/W) CDs, R/W DVDs, etc. Other types of storage devices, memories, and media are also included herein.

The non-transitory data storage 164 may also store a set of program instructions 166 and a speech recognition system 167. The program instructions 166 are executable by the processor 162 to perform various operations, such as the operations described in this disclosure. The at least one processor 162 can include one or more processors, such as one or more general-purpose microprocessors and/or one or more special purpose microprocessors. The one or more processors may include, for instance, an application-specific integrated circuit (ASIC) or a field-programmable gate array (FPGA). Other types of processors, computers, or devices configured to carry out software instructions are also contemplated herein. The controller 160 may also include a Wi-Fi module. The Wi-Fi module can enable the system 100 to have internet connectivity. The processor 162 may provide instructions to the Wi-Fi module.

The speech recognition system 167 can be a technology that enables computers to understand and interpret spoken language. It can work by converting spoken words or phrases into text, allowing users to interact with devices using voice commands. The speech recognition system 167 typically comprises three main components: an audio input device such as a microphone, a speech recognition engine, and natural language processing algorithms. When a user speaks into the microphone, the audio input is captured and processed by the speech recognition engine, which analyzes the waveform to identify individual phonetic units and words. The engine then matches these units against a database of known words and language models, using statistical techniques and machine learning algorithms to determine the most probable transcription for the spoken words. Finally, the transcribed text is outputted as written text or used to execute commands within a computer system.

Referring back to FIG. 1, in one implementation the physiological signal monitor 120, the microphone device 140, and the controller 160 are all in communication but are not part of the same device. For example, the physiological signal monitor 120, the microphone device 140, and the controller 160 could all be part of the same network. Information and instructions could be wirelessly transferred between the physiological signal monitor 120, the microphone device 140, and the controller 160 to execute various principles described herein. The information can be transferred over Wi-Fi. In one implementation, the microphone may be part of a badge worn by the user and my transmit audio to the controller 140 to annotate data from the physiological signal monitor 120.

In an alternative implementation, the system 100 is a device. The physiological signal monitor 120, the microphone device 140, and the controller 160 can all be components of the device. For example, they could all be electrically connected and integrated into the device, in one housing. The physiological signal monitor 120, the microphone device 140 and controller 120 could be used in the device to execute various principles described herein. For example, the microphone device 140 within the device can be used to gather audio data, the physiological device 120 can be used to gather patient data, and the controller 160 can process the audio data and patient data to determine further steps to execute. All of this could be accomplished at the device. For example, in an example implementation, the physiological signal monitor 120 is a defibrillator and the microphone device 140 and the controller 160 could be integrated into the defibrillator.

A defibrillator is a physiological signal monitor used to treat life-threatening cardiac arrhythmias, specifically ventricular fibrillation and pulseless ventricular tachycardia, which can lead to sudden cardiac arrest. The defibrillator works by delivering an electric shock to the heart, which aims to reset the heart's electrical system and restore a normal rhythm. To use a defibrillator, electrode pads are placed on the patient's chest, allowing the device to monitor the heart's electrical activity. The defibrillator's built-in computer continuously analyzes the heart's rhythm and determines whether a shock is necessary. If a shockable rhythm is detected, meaning the heart is in ventricular fibrillation or pulseless ventricular tachycardia, the defibrillator charges up to deliver a high-energy electric shock to the heart.

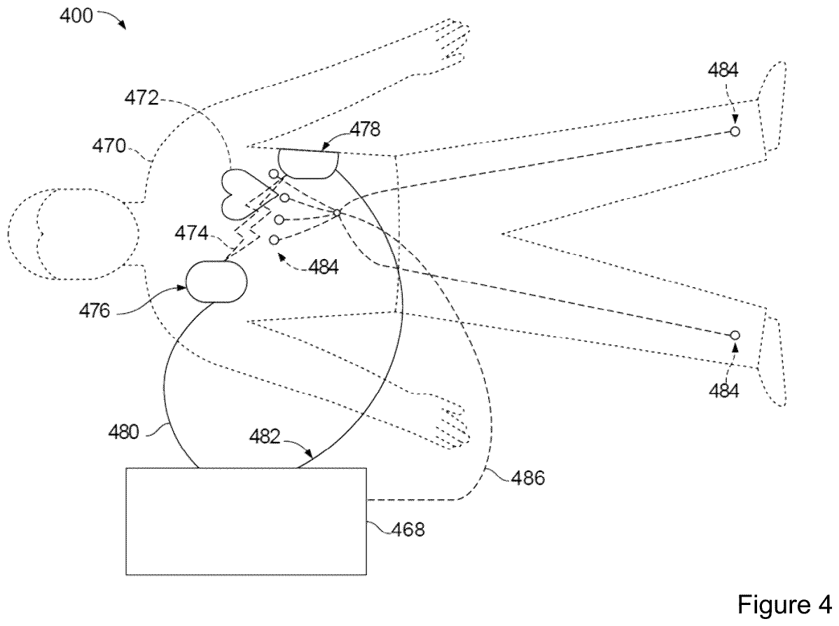

FIG. 4 is a diagram of a representation of an exemplary scene 400 showing use of a defibrillator 468 for monitoring and providing treatment or therapy to a person 470 experiencing a medical condition, such as a cardiac arrest. The defibrillator 468 may be operated by a user (e.g., a healthcare professional, service worker, a doctor, a first responder, etc.) and may be used in a hospital or a pre-hospital environment or setting. The defibrillator 468 may include functions and operations of a manual defibrillator, an automatic defibrillator (AED), or any other suitable defibrillator. In some examples, the defibrillator 468 may be a monitor defibrillator, which is a combination of a monitor and a defibrillator.

As shown in FIG. 4, the defibrillator 468 is positioned near the person 470 (e.g., patient). The person 470 may be experiencing a condition in his or her heart 472, which could be, for example, cardiac arrest or any other cardiac rhythm abnormality. The person 470 may be lying on his or her back on a surface, such as the ground or a bed, and may be located in a hospital, a home, or a pre-hospital environment (e.g., an ambulance). The defibrillator 468 may be configured to generate an electrical pulse 474 and deliver the electrical pulse 474 to the person 470. The electrical pulse 474, also known as a defibrillation shock or therapy shock, is intended to go through the chest of person 470 and restart the heart 472, in an effort to save the life of person 470. The electrical pulse 474 can further include one or more pacing pulses and the like.

The electrical pulse 474 may be delivered to the person 470 using defibrillation electrodes 476 and 478. The defibrillation electrodes 476 and 478 may include hand-held electrode paddles or electrode pads placed on the body of the person 470. An electrical cable 480 may connect the defibrillation electrode 476 to the defibrillator 468 and an electrical cable 482 may connect the defibrillation electrode 478 to the defibrillator 468. When the defibrillation electrodes 476 and 478 are in electrical contact with the body of person 470, the defibrillator 468 may administer, via the defibrillation electrodes 476 and 478, the electric pulse 408 through the heart 472 of person 470. The defibrillation electrodes 476 and 478 may also be configured to sense or detect one or more physiological parameters of the person 470 and to generate signals representative of the physiological parameters.

As shown in phantom in FIG. 4, one or more sensors 484 may be placed at various locations on the body of the person 470. In an example implementation, at least some of sensors 484 can be electrodes. The sensors 484 may be configured to sense or detect physiological parameters of the person 470 and to produce signals representative of the physiological parameters. The sensors 484 can be removably coupled to the defibrillator 468. In an example implementation, the sensors 484 could be ECG sensors. The sensors 484 could also include impedance sensors, temperature sensors, O2 sensors, blood pressure sensors, heart rate sensors, and CO2 sensors.

An electrical cable 486 may connect the sensors 484 to the defibrillator 468. Alternatively, the sensors 484 could be in wireless communication with the defibrillator 468. The physiological parameters generated by the sensors 484 and/or the defibrillation electrodes 476 and 478 may be provided to the defibrillator 468 for analysis. The physiological parameters may include ECG data, heart rhythm data, heart rate data, cardiac output data, blood flow data, a level of perfusion, respiration data, body temperature data, and/or any other suitable physiological parameter that may be used to assess the physical condition of the person 470.

The defibrillator 468 may be configured to select an appropriate treatment protocol based on the physiological parameters. For example, the defibrillator 468 may determine a cardiopulmonary resuscitation (CPR) treatment protocol to apply to the person 470. The defibrillator 468 may also determine whether a defibrillation pulse should be applied or delivered to the person 470. Further, the defibrillator 468 may display the representations of the physiological parameters of the person to assist a user in treating and diagnosing medical conditions. In an example implementation, the physiological parameters can include ECG data, invasive blood pressure, CO2, SpO2, non-invasive blood pressure, and temperature. The physiological parameters may be displayed in the order of ECG waveforms at the top, followed by invasive blood pressure, followed by EtCO2, followed by non-invasive blood pressure, followed by temperature.

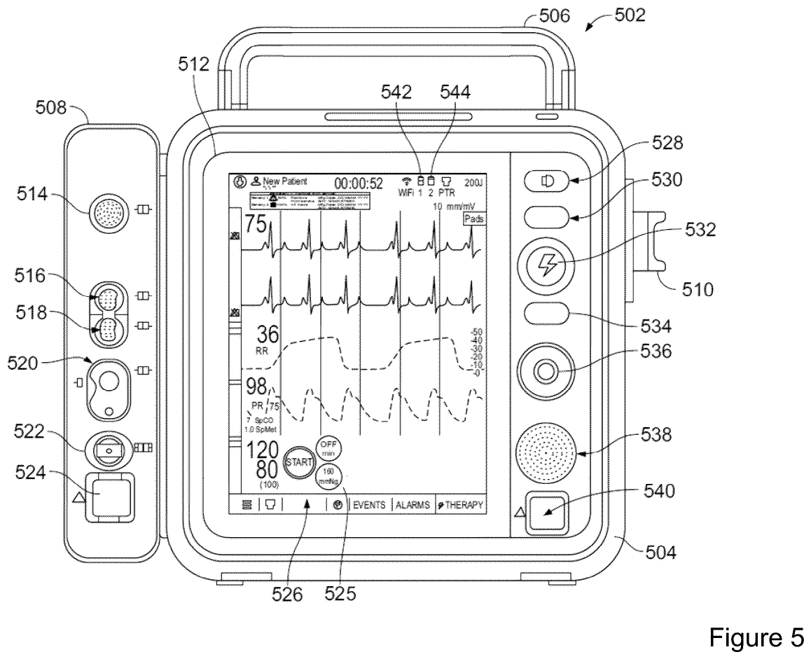

FIG. 5 illustrates a front view of a defibrillator 502, in accordance with an example implementation. The defibrillator 502 can comprise the defibrillator 468 of FIG. 4. The defibrillator 502 may be configured to monitor and provide treatment or care to a person or patient experiencing a medical condition, such as a cardiac arrest or any other cardiac rhythm abnormality. The defibrillator 502 may be operated by a user (e.g., a medical professional, a first responder, a healthcare professional, service worker, a doctor, etc.) and may be used in a hospital or a pre-hospital environment or setting. As illustrated, the defibrillator 502 may be a monitor defibrillator, which is a combination of a monitor and a defibrillator.

As a defibrillator, the defibrillator 502 may be configured to deliver an electrical pulse or shock to a person experiencing a medical condition. The defibrillator 502 may be configured to operate or function in one or more defibrillation modes, such as a manual defibrillation mode, an AED mode, or any other suitable defibrillation mode. For example, the defibrillator 502 may be selected to operate in an AED mode when the defibrillator 502 is intended to be used by first responders and/or people who are not trained in providing medical treatment using defibrillation. When operating in the AED mode, the defibrillator 502 can determine whether a defibrillation pulse or shock is needed and, if so, charge an energy storage device (e.g., a capacitor) of the defibrillator 502 to a predetermined energy level and instruct the user to administer the defibrillation shock. The defibrillator 502 may also be selected to operate in a manual defibrillation mode when the defibrillator 502 is intended to be used by persons who are trained to provide medical treatment using defibrillation (e.g., medical professionals, such as doctors, nurses, paramedics, emergency medical technicians, etc.). When the defibrillator 502 is configured to operate in the manual defibrillation mode, the device provides an array of patient vital signs to the user, including the patient's ECG data, allowing user interpretation of the patient vital signs, and allows the user to change the selected energy (although configuration options may set a default energy), charge the defibrillator energy storage capacitor, and deliver a shock to the patient.

As a monitor, the defibrillator 502 can monitor and evaluate physiological parameters of a person being treated for a medical condition. The defibrillator 502 may receive or acquire signals or voltage from one or more electrodes or sensors placed at various locations on the body of the person. The electrodes may sense or detect the physiological parameters of the person and produce signals representative of the physiological parameters. The representations of the physiological parameters may be displayed by the defibrillator 502 to assist a user in treating and diagnosing medical conditions of a person. The physiological parameters may include ECG data, body temperature, non-invasive blood pressure (NIBP), SpO2 data, a concentration or partial pressure of carbon dioxide in the respiratory gases (e.g., capnography), and/or any other suitable physiological parameter of a person. These physiological parameters of the person can be stored in the memory of the defibrillator 502. In some examples, the physiological parameters may be transmitted to other remote communication or computing devices and databases.

As shown in FIG. 5, the defibrillator 502 includes a housing or casing 504, a handle 506, an input module or interface 508, a defibrillation interface 510, and a user interface 512. The handle 506 of the defibrillator 502 is attached to the housing 504 to allow a user to move or transport the defibrillator 502 to a location near a person experiencing a medical condition. The housing 504 may be generally rectangular or square shaped. In other examples, the housing 504 can have any other suitable shapes. The housing 504 may be formed from various materials or combinations of materials. For example, the housing 504 may be made of molded plastic, metal, or some combination of both.

The input module 508 of the defibrillator 502 may be coupled to or integral with the housing 504. The input module 508 may enable the defibrillator 502 to receive vital signs and other physiological parameters (e.g., a heart rate (HR) and ECG data) of a person via sensors (e.g., the sensors 118 of FIG. 4). The sensors may be placed on the body of a person to sense or detect signals generated by the body or heart of the person. The input module 508 may include one or more ports or receptacles to enable the sensors to be detachably coupled to the input module 508. As shown in FIG. 5, the input module 508 can include a port 514 configured to be connected to an oxygen saturation (SpO2) sensor, a port 516 configured to be connected to a temperature sensor, a port 518 configured to be connected to a sensor for measuring invasive blood pressure (IP) via a catheter, a port 520 configured to be connected to a sensor for measuring partial pressure of carbon dioxide (CO2) in gases in the airway via capnography, and a port 522 configured to be connected to sensor for measuring a non-invasive blood pressure (NIBP). The input module 508 may also include a communication port 524, such as a Universal Serial Bus (USB) port, which can be used to connect to an input device (e.g., a mouse, a keyboard, etc.). The input module 508 may include other ports to enable any other suitable physiologic parameter of a person to be monitored and evaluated by the defibrillator 502.

The defibrillation interface 510 of the defibrillator 502 is configured to enable electrical charges or pulses to be delivered or applied to a person via defibrillation electrodes (e.g., the defibrillation electrodes 476 and 478 of FIG. 4). The defibrillation electrodes may also be configured to enable the defibrillator 502 to receive physiological parameters of a person (e.g., a heart rate (HR), ECG data, etc.). The defibrillation interface 510 may include one or more ports or receptacles capable of allowing defibrillation electrodes to be detachably coupled to the defibrillation interface 510. A cable assembly or therapy cable may enable the defibrillation electrodes to be coupled to the ports of the defibrillation interface 510. Each therapy cable may include a defibrillation electrode attached at one end and a connector attached to the other end. The connector may be configured to be coupled to or plugged into a port or receptacle of the defibrillation interface 510.

The user interface 512 of the defibrillator 502 may include one or more input devices configured to receive inputs or commands from a user and one or more output devices configured to provide information to the user. The input devices of the user interface may include various controls, such as electronic or mechanical switches, pushbuttons, keyboards, touchscreens, microphones, scanners, and/or cameras, etc., for operating the defibrillator 502. The output devices of the user interface 512 can be visual, audible or tactile, for communicating to a user of the defibrillator 502 (e.g., a medical professional, a first responder, etc.). For example, the output devices may be configured to present visual alarms or alerts, flashing lights, and/or warnings to the user. The output devices may also include an audio system that provides audio signals to aurally communicate with the user voice prompts that deliver instructions or commands, monotonal, ascending, descending or quickening tones to indicate alerts or warnings, or any other suitable audio signals for communicating with the user of the defibrillator 502.

As shown in FIG. 5, the user interface 512 of the defibrillator 502 includes a power button 528 configured to turn the defibrillator on and off (e.g., “On-Off” button), a charge button 530 configured to cause the defibrillator 502 to build an electric charge to be applied to the person in a form of a defibrillation shock or pulse, a defibrillation shock button 532 configured to cause the defibrillator 502 to apply a defibrillation pulse or therapy shock to a person during a defibrillation episode, an analyze button 534 configured to cause the defibrillator 502 to analyze physiologic parameters of a person (e.g., ECG data) to facilitate determining the appropriate time to apply a defibrillation pulse or shock, and a speed dial button 536 configured to navigate through menus of on-screen software. The user interface 512 may also include a speaker 538 to provide audio output and a USB output port 540 to facilitate connecting the defibrillator 502 to a device such as a printer. The user interface 512 may include any other suitable output device for providing outputs or input device for receiving inputs from a user.

As shown in FIG. 5, the user interface 512 of the defibrillator 502 may also include a display device 525 (e.g., a touchscreen) for displaying a graphical user interface (“GUI”) 526. The GUI 526 can display physiologic parameters of a person (e.g., a patient), provide visual feedback to the user (e.g., a healthcare provider) about a condition of the person, provide information about the defibrillator 502, and allow the user to interact with and operate the defibrillator 502. The GUI 526 may also be configured to visually present various measured or calculated parameters associated with the person indicating the physical status of the person (e.g., an ECG), and/or instructions and/or commands, including prompts to perform cardiopulmonary resuscitation (CPR) treatment or other treatment instructions, to the user. Further, the GUI 526 may include multiple visual user interface items that are selectable or “clickable” by the user including user-selectable icons, user-selectable on-screen buttons, menus, widgets, scroll bars, graphical objects, and other items for facilitating user interaction.

As shown in FIG. 5, the GUI 526 includes graphical representations of a first battery unit indicator 542 and a second battery unit indicator 544. The first battery unit indicator 542 and second battery unit indicator 544 are configured to display a status of a first battery unit and a status of the second battery unit, respectively. For example, the first and second battery unit indicators 542 and 544 may provide a charging level of the first and second battery units. The GUI 526 may also display messages and information about the end of life of a first battery unit and/or a second battery unit. As shown, the GUI 526 may indicate that the first battery unit should be removed from service. The GUI 526 may also provide additional information about the battery units of the defibrillator 502, such as battery charge levels (e.g., a remaining charge), manufacturing dates, serial numbers, etc. Further, the GUI 526 may provide audible alarms or warnings when a battery unit of the defibrillator 502 has reached its end of life and/or should be removed and replaced.

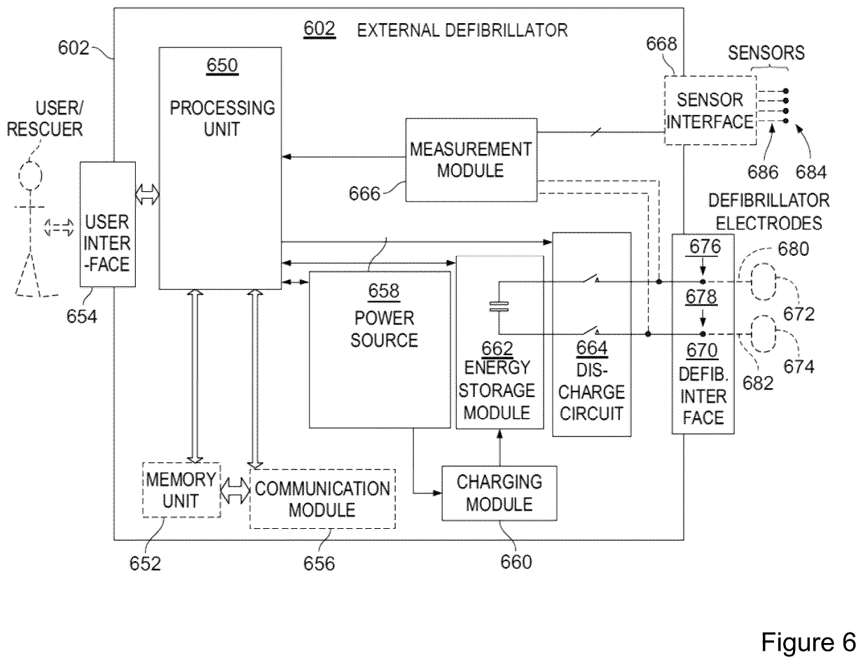

FIG. 6 is a diagram showing components of a defibrillator 602, in accordance with an exemplary implementation. As shown in FIG. 6, the defibrillator 602 includes a processing unit 650, a memory unit 652, a user interface 654, a communication module or interface 656, a power source 658, a charging module 660, an energy storage module or device 662, a discharge circuit 664, a measurement module 666, a sensor interface 668, and a defibrillator interface 670. These components can be, for example, included in the defibrillators of FIG. 4 or 5.

The defibrillation interface 670 of the defibrillator 602 may be configured to enable an electrical pulse or shock to be delivered or applied to a person (e.g., a patient) experiencing a medical condition. The defibrillation interface 670 may include the defibrillation interface 510 of FIG. 5. The defibrillation interface 670 may include one or more ports or nodes 676 and 678 to enable the defibrillation electrodes 672 and 674 to be detachably coupled to the defibrillation interface 670. A cable assembly or therapy cable 680 may enable a defibrillation electrode 672 to be coupled to the port 676 of the defibrillation interface 670 and a cable assembly or therapy cable 682 may enable a defibrillation electrode 674 to be coupled to the port 678 of the defibrillation interface 670 of the defibrillator 602. The connectors of the cable assemblies 680 and 682 may be configured to be coupled to or plugged into the ports 676 and 678 of the defibrillation interface 670. The connectors can be male or female connectors that are compatible with the ports 676 and 678 of the defibrillation interface 670 of the defibrillator 602.

The defibrillation interface 670 may also enable the defibrillator 602 to receive physiological parameters (e.g., a heart rate (HR) and ECG data) of the person from the defibrillation electrodes 672 and 674. These physiological parameters can be stored in a patient electronic health record. Each of the defibrillation electrodes 672 and 674 may be configured to measure one or more physiological parameters of the person (e.g., heart electrical activity, heart rate, etc.) and to provide signals representative of the physiological parameters to the defibrillation interface 670. For example, when the defibrillation electrodes 672 and 674 are placed on the chest of the person, an ECG signal of the person can be detected as a voltage difference between the defibrillation electrodes 672 and 674. The defibrillation electrodes 672 and 674 may also be used to determine device parameters indicative of a condition of the defibrillator, such as an electrode impedance or a user interaction with the defibrillator 602. For example, the defibrillator 602 can detect an impedance between the defibrillation electrodes 672 and 674 to determine whether the defibrillation electrodes 672 and 674 are making sufficient electrical contact with a person's body.

The sensor interface 668 of the defibrillator 602 may also be configured to receive physiological parameters (e.g., a heart rate (HR) and ECG data) from one or more sensors 684. The sensors 684 may be placed in contact with the body of a person being monitored or treated for a medical condition. Each of the sensors 684 may include a transducer configured to sense a signal or voltage of the person. For example, the sensors 684 may measure or detect heart electrical activity, such as an electrocardiogram (ECG), saturation of the hemoglobin in arterial blood with oxygen (SpO2), carbon monoxide (carboxyhemoglobin, COHb) and/or methemoglobin (SpMet), partial pressure of carbon dioxide (CO2) in gases in the airway by means of capnography, total air pressure in the airway, flow rate or volume of air moving in and out of the airway, blood flow, blood pressure (e.g., non-invasive blood pressure (NIBP)), core body temperature with a temperature probe in the esophagus, oxygenation of hemoglobin within a volume of tissue (rSO2), and any other physiological parameters of a person being monitored or treated.

The sensor interface 668 may include one or more receptacles or ports (e.g., an ECG port) to enable the sensors 684 (e.g., physiological sensors) to be detachably coupled to the defibrillator 602. The sensors 684 may be attached to the sensor interface 668 by cable assembles or therapy cables 686. In some implementations, the sensors 684 may be fixedly connected to the sensor interface 668. The therapy cables 686 may each include a sensor 684 at one end and to a connector at the opposite end. The connector can be configured to be coupled to or plugged into a port or receptacle of the sensor interface 668.

The measurement module 666 of the defibrillator 602 may be configured to receive signals or sensor data from the sensor interface 668 and the defibrillation interface 670. The signals may be representative of physiological parameters of a person being monitored and/or treated for a medical condition. The measurement module 666 may measure or determine various physiological parameters from the signals. For example, the measurement module 666 may determine an ECG of the person based on a voltage difference between the defibrillation electrodes 672 and 674. The measurement module 666 may also determine device parameters indicative of a condition of the defibrillator 602 such as an electrode impedance or a user interaction with the defibrillator 602. For example, the measurement module 666 may measure an impedance or voltage across the defibrillation electrodes 672 and 674 or a pair of sensor.

The measurement module 666 may also include a filter circuit or hardware (e.g., amplifiers, filters, etc.) to attenuate and/or filter at least some of the noise that may be present on the signals received from the sensor interface 668 and/or the defibrillation interface 670. For example, the filter circuit may apply at least one filter to the signal to remove artifacts resulting from chest compressions being delivered to the person. In some implementations, the filter may be implemented as an analog filter, a digital filter, or combinations of both. The measurement module 666 may also digitize the signals received from the sensor interface 668 and/or defibrillation interface 670 prior to transmitting the signals to the processing unit 650.

The memory unit 652 of the defibrillator 602 is in operable communication with the processing unit 650. The memory unit 652 may store various values, look-up tables, equations, audio and video files, and/or plurality of treatment protocols that can be read and accessed by the processing unit 650. The treatment protocols may include instructions regarding CPR treatment (e.g., chest compressions). The memory unit 652 can also store a person's sensed physiological parameters, historical data, lengths of time and rate of CPR treatments, and defibrillation pulses previously discharged. Further, the memory unit 652 can store instructions or computer executable code of software routines that can be retrieved and executed by the processing unit 650.

The memory unit 652 may include one or more computer-readable storage media that can be read or accessed by the processing unit 650. The computer-readable storage media can include volatile and/or non-volatile memory, dynamic random-access memory (DRAM) read-only memory (ROM), random access memory (RAM), magnetic disk storage media, optical storage media, smart cards, flash memory devices, or any other suitable memory. The computer-readable storage media can be integrated in whole or in part with the processing unit 650. The computer-readable storage media may be implemented using a single physical device (e.g., one optical, magnetic, organic or other memory or disc storage unit), while in other examples, the computer readable storage media can be implemented using two or more physical devices.

The processing unit 650 of the defibrillator 602 is configured to control various operations of the defibrillator 602. The processing unit 650 may receive inputs from various components of the defibrillator 602 and process the inputs to produce outputs that may be stored in the memory unit 652 and/or displayed on the user interface 654. For example, the processing unit 650 may receive and evaluate physiological parameters of a person (e.g., electrical activity of the heart) from the sensors 684 and/or the defibrillation electrodes 672 and 674 placed on a person. The processing unit 650 may also determine whether a defibrillation pulse should be delivered to a person based upon the physiological parameters. For example, the processing unit 650 may make a shock/no shock determination based on ECG data or other information. In some examples, the processing unit 650 may cause the defibrillator 602 to automatically deliver defibrillation pulses in AED mode or may advise the user of these determinations via the user interface 654. When a defibrillation shock has previously been delivered, the processing unit 650 may evaluate the efficacy of the delivered defibrillation pulse by determining if the heart of the person is still fibrillating based on the sensed electrical activity in order to determine whether an additional defibrillation pulse is warranted.

The processing unit 650 may also determine whether to commence charging of the energy storage module 662. This processing unit 650 may determine the rate to charge the energy storage module 662. Further, in manual mode, the processing unit may cause an electrocardiogram (ECG) to be displayed on the user interface 654 that reflects the sensed electrical activity of a heart of a person. In addition, the processing unit 650 may control delivery of other types of treatment therapy to the person via the defibrillation electrode 672 and 674, such as cardioversion or pacing therapy.

The processing unit 650 may include one or more general-purpose processors, special purpose processors (e.g., digital signal processors (DSP), application specific integrated circuits (ASIC), graphic processing units, etc.), or any other suitable processing unit or controller. The processing unit 650 can be configured to execute instructions (e.g., computer-readable program instructions) that are stored in memory and may be executable to provide the functionality of the defibrillator described herein. For example, the processing unit 650 can execute instructions for causing the defibrillator to display an ECG waveform on the user interface 654 of the defibrillator 602.

The user interface 654 of the defibrillator 602 facilitates user interactions with the defibrillator 602. The user interface may comprise the GUI 526 of FIG. 5. The user interface 654 may include various types of input devices for receiving inputs or commands from a user. For example, the input devices may include keyboards, electrical or mechanical switches, microphones, pushbuttons, touchscreens, scanners, and/or any other suitable input device for enabling inputs to the defibrillator 602. For instance, a user can use interface items displayed on the GUI to input information regarding a particular event (e.g., a treatment or medication administered to the person). Additionally, the user interface can be used to rearrange the waveforms displayed on the defibrillator 602, as described in further detail below.

The user interface 654 may also comprise various types of output devices for providing information to the user. The output devices of the user interface can be visual, audible or tactile for communicating to or providing feedback to a user (e.g., a rescuer, a first responder, a healthcare professional, etc.). The output devices may include a screen, one or more light emitting diodes (LEDs), and/or a speaker to output various sounds (e.g., voice or audio), etc. For example, the output device can also be configured to present visual alarms or alerts, flashing lights, and/or warnings to the user of the defibrillator. The output device may also include an audio system that provides an audio signal to aurally communicate with user voice prompts that deliver instructions or commands, monotonal, ascending, descending or quickening tones to indicate alerts or warnings, or any other suitable output device for communicating with the user.

The communication module 656 of the defibrillator 602 may be in communication with the processing unit 650. The communication module 656 may enable patient data, treatment information, CPR performance, system data, environmental data, etc. to be communicated between the defibrillator 602 and other devices, such as a remote assistance center and/or any other remote computing device. The communication module 656 may include one or more wireless or wireline interfaces that allow for both short-range communication and long-range communication to one or more networks or to one or more remote devices. The wireless interfaces may provide for communication under one or more wireless communication protocols, such as Bluetooth, Wi-Fi (e.g., an institute of electrical and electronic engineers (IEEE) 802.xx protocol), Long-Term Evolution (LTE), cellular communications, near-field communication (NFC), radio-frequency identification (RFID), and/or other wireless communication protocols. Such wireline interfaces may include an Ethernet interface, USB interface, or similar interface to communicate via a wire, a twisted pair of wires, a coaxial cable, an optical link, a fiber-optic link, or other physical connection to a wireline network.

The power source 658 of the defibrillator 602 may provide power to the components of the defibrillator 602. The power source 658 may include one or more battery units. The battery units may include lithium batteries, alkaline batteries, nickel cadmium batteries, nickel metal hydride batteries, or any other suitable type of battery or energy device.

The charging module 660 of the defibrillator 602 may be configured to charge the energy storage module or device 662 before a defibrillation shock is delivered to a person. The charging module 660 may include charging circuitry, such as a flyback charger, for charging the energy storage module 662 to a selected voltage level that is determined based on a selected energy level for the defibrillation shock. During operation of the defibrillator 602, the charging module 660 may draw power from the power source 658 to charge the energy storage module 662.

The energy storage module 662 of the defibrillator 602 can store electrical energy in the form of an electrical charge, for delivery of a defibrillation pulse or shock to a person being treated for a medical condition. The energy storage module 662 can be charged by the charging module 660 to a desired energy level (e.g., between 50 Joules to 660), as controlled by processing unit 650. For instance, for an adult, the processing unit 650 can select an energy level from an adult energy sequence that includes energy levels of 200 Joules, 300 Joules, and 360 Joules. For a pediatric patient, the processing unit 650 can select an energy level from a pediatric energy sequence that includes energy levels of 50 Joules, 75 Joules, and 90 Joules. In some implementations, the energy storage module 662 may include one or more capacitors that stores the energy to be delivered to a person as a defibrillation shock. When the energy of the energy storage module 662 reaches the desired energy level, the defibrillation shock may be delivered manually or automatically. For example, the user interface 654 may provide an indication to the user that defibrillator 602 is ready to deliver a defibrillation pulse or shock.

The discharge circuit 664 of the defibrillator can enable the energy stored in the energy storage module 662 to be discharged to a person. The electrical energy may be discharged at a selected energy level, via the ports or nodes 676 and 678, to the defibrillation electrodes 604, 608 to cause a shock to be delivered to the person. The discharge circuit 664 can be controlled by the processing unit 650, or directly by the user via the user interface 654. In some implementations, the discharge circuit 655 can include one or more switches that, when activated, couple the energy storage module 662 to the ports or nodes 676 and 678 to deliver a defibrillation shock to person via defibrillation electrodes 672 and 674. For example, the processing unit 650 may activate the switches to electrically connect energy storage module 662 to the defibrillation electrodes 672 and 674, and thereby deliver the defibrillation shock to person. The switches can be made in a number of ways and may be formed, for example, of electrically operated relays. Alternatively, the switch may comprise an arrangement of solid-state devices such as silicon-controlled rectifiers or insulated gate bipolar transistors.

In one implementation, the aforementioned system 100 can be used to carry out operations. The operations can include listening to speech uttered by a user, and detecting an action trigger present in the speech uttered by the user. In response to detecting the action trigger, the operations can include obtaining an audio data segment including speech from the user. The operations can further include determining that the audio data segment including speech from the user includes a physiological signal monitor command, determining if the physiological signal monitor command is valid, and instructing the physiological signal monitor to perform a physiological signal monitor action associated with the physiological signal monitor command. The physiological signal monitor action could be annotating data gathered by the physiological signal monitor, annotating a patient electronic health record, or performing an action on the patient with the physiological signal monitor.

In one implementation, the microphone device 140 can listen to speech uttered by a user. The user may be a healthcare provider or a user of the system 100 for emergency treatment. The microphone device 140 may be in a default passive listening mode. The microphone device 140 can capture acoustic waves produced by the user's vocal cords and surrounding environment. When a user speaks, their vocal cords vibrate, producing sound waves that propagate through the air. The microphone's diaphragm responds to these variations in air pressure, converting them into electrical signals. These signals, representing the speech waveform, are then transmitted to the controller 160, which may include analog or digital signal processing algorithms designed to filter, enhance, and analyze the audio data.

The controller 160 may detect an action trigger present in the speech uttered by the user. The action trigger may be a specific word, phrase, or pattern recognized by the speech recognition system or natural language processing algorithm that initiates a predefined action or response. For example, the action trigger could be a wake word, or a command for the system 100 to carry out. In one implementation, the controller 160 may also recognize synonyms for the specific word or phrase used as the action trigger.

In response to detecting the action trigger, the microphone 140 may continue to listen to the speech uttered by the user so as to obtain an audio data segment to send to the controller 160. The microphone 140 may listen to the speech uttered by the user to obtain the audio data segment for a predetermined duration. For example, the microphone may only obtain an audio data segment that is 30 seconds long, one minute long, or two minutes long. Alternatively, the microphone 140 may listen to the speech uttered by the user until the user is finished speaking. The duration of the audio data segment may be determined by when the user is done speaking. The controller 160 may put the audio data segment through the speech recognition system or a natural language processing algorithm to determine the content of the audio data segment.

The controller 160 may then determine that the audio data segment comprising speech from the user includes a physiological signal monitor command. The physiological signal monitor command may be a command for the physiological signal monitor to initiate an action. For example, the user could speak a command to the physiological signal monitor for it to perform an action on a patient. As previously mentioned, the physiological signal monitor could be a defibrillator. In this implementation, the physiological signal monitor command could be at least one of charge, shock, annotate, or display. In other words, the user could audibly command the defibrillator to charge, to administer a shock to the patient, to add annotation to patient data that the defibrillator has gathered, or to display something. When annotating data, the physiological signal monitor could add a note to the patient record, or could annotate physiological data previously gathered from the patient.

Before executing the command, the controller 160 may first determine if the physiological signal monitor command is valid. In other words, the controller 160 may take extra precautions in order to determine that a command really was given by the user. This is to avoid mistakes in any emergency situations. Determining if the physiological signal monitor command is valid can include determining whether the controller 160 might have falsely interpreted the audio data segment. In order to determine if the physiological signal monitor command is valid, the controller 140 may require voice confirmation of the physiological signal monitor command and the microphone device 160 may obtain the voice confirmation. For example, after the command has been received, the system 100 may request voice confirmation of the command by restating the command as the physiological signal monitor understood it. This could be achieved by issuing an audio request for the user to verbally confirm the command (e.g., playing a request for confirmation out of speakers on the physiological signal monitor), or by visually displaying a request for a verbal confirmation of the command on the graphical user interface of the physiological signal monitor. Once the request has been issued, the system 100 may use the microphone 160 to listen for an answer in order to obtain the voice confirmation of the physiological signal monitor command. The controller 140 may determine if the voice confirmation is positive or negative based on the answer obtained by the microphone 160.

In one implementation, the system 100 may seek voice confirmation of the physiological signal monitor command every time the controller 140 determines that the audio data segment comprising speech from the user includes a physiological signal monitor command. Alternatively, the system 100 may seek voice confirmation of the physiological signal monitor command only when the importance of the command is over a threshold importance. For example, certain commands may be more important than others. The commands may be classified by levels of importance. Any command that is level 2 or greater importance, may require verbal conformation. Examples of level 2 or greater importance commands could be changing a patient's therapy, removing a “critical parameter” from the graphical user interface, or powering the physiological signal monitor down.

In one implementation, once the controller 140 determines that the physiological signal monitor command is valid using the aforementioned techniques, the controller 140 can request a confirmation command from the user. The confirmation command could be at least one of a voice confirmation or a touch confirmation. The voice confirmation could be the user verbally giving confirmation of the command which the microphone 160 listens. This could be achieved by the user giving a verbal confirmation such as “yes,” “activate,” or “confirmed.” Alternatively, the touch confirmation could include the user selecting a confirmation button on the graphical user interface of the physiological signal monitor 120.

The controller 140 could also request a confirmation command from the user after performing a review of patient parameters. For example, the controller 140 could check the patient's age and weight in response to receiving a command to administer a drug. If the patient's age and weight do not match with the guidelines for administering the drug, the controller 140 could request a confirmation command.

The controller 140 could alternatively determine that the physiological signal monitor command is invalid using the aforementioned techniques. In response to determining that the physiological signal monitor command is invalid, the controller 140 could request a further command from the user. This could be an additional request after attempting the validate the initial command. For example, the system 100 could issue an audible alert or display an alert on the GUI. The alert could ask if the user is sure about the command, state that there is an unrecognized command, or request that the user use a physical button.

Once the controller 140 determines that the physiological signal monitor command is valid, the controller 140 could instruct the physiological signal monitor to perform a physiological signal monitor action associated with the physiological signal monitor command. There could be a list stored within the controller 140 that associates physiological signal monitor commands, and any synonyms, with physiological signal monitor actions. The physiological signal monitor actions could be anything from annotating a patient record stored on the physiological signal monitor or data stored on the physiological signal monitor, to taking a therapeutic action on a patient. Annotating the patient record could include adding a note to the patient record, or adding a note to the data gathered by the physiological signal monitor. The note could be the occurrence of an event, a user's observation, or anything else that might have happened during treatment. The therapeutic action could be any operation that the physiological signal monitor is configured to perform on a patient. For example, if the physiological signal monitor is a defibrillator, the action could be to start charging, to deliver a shock, or to annotate the patient record with a note that epinephrine was administered at a specific time.

### III. Example Methods

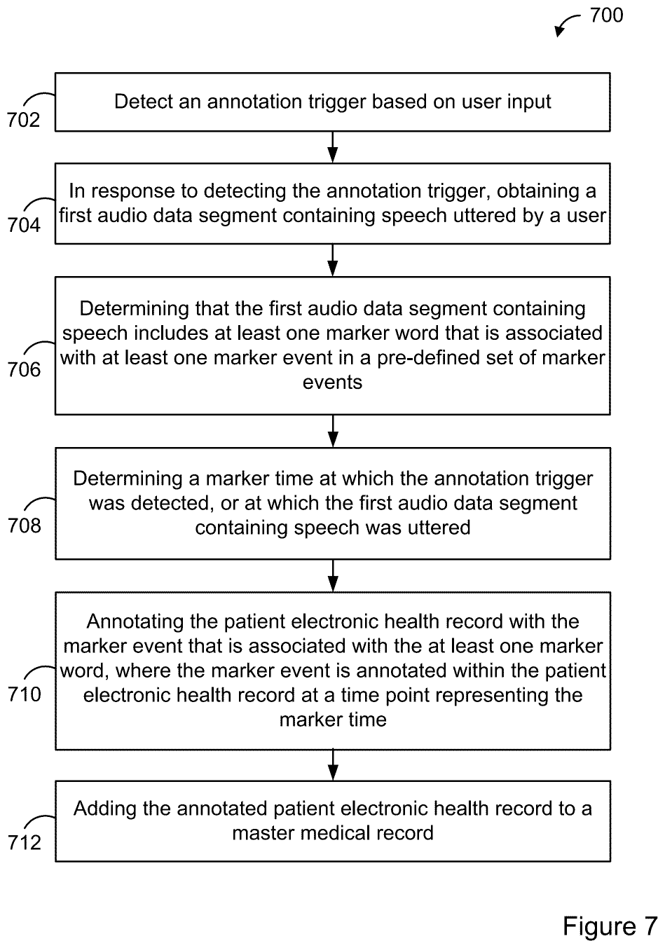

FIG. 7 illustrates a flow chart of a method 700 for annotating a patient electronic health record. The method 700 represents an example method that may include one or more operations, functions, or actions, as depicted by one or more of blocks 702-712, each of which may be performed by any of the systems or defibrillators described herein. For instance, the system depicted in FIG. 1 and/or the defibrillator depicted in FIGS. 5 and 6 may enable execution of method 700.

Those skilled in the art will understand that the flowchart of FIG. 7 herein illustrates functionality and operations of certain implementations of the present disclosure. In this regard, each block of the flowchart may represent a module, a segment, or a portion of program code, which includes one or more instructions executable by one or more processors for implementing specific logical functions or steps in the process. The program code may be stored on any type of computer readable medium, for example, such as a storage device including a disk or hard drive.

In addition, each block may represent circuitry that is wired to perform the specific logical functions in the process. Alternative implementations are included within the scope of the example implementations of the present application in which functions may be executed out of order from that shown or discussed, including substantially concurrent or in reverse order, depending on the functionality involved, as would be understood by those reasonably skilled in the art.

The method illustrated in FIG. 7, can include detecting a user input, such as an annotation trigger. In response to detecting the annotation trigger, the method can include obtaining a first audio data segment containing speech uttered by a user. The method can then include determining that the first audio data segment includes at least one marker word. The marker word may be associated with at least one marker event that is in a pre-defined set of marker event. The method can then include determining a marker time. The marker time can be the time at which the annotation trigger was detected, or at which the first audio data segment containing speech was uttered. The method can also include annotating the patient electronic health record with the marker event at a time point representing the marker time. The patient electronic health record can be based on data generated by the physiological signal monitor. The method can further include adding the annotated patient electronic health record to a master medical record. This implementation can allow a user to annotate a patient electronic health record without needing to use their hands. In emergency situations, this can be particularly beneficial.

Block 702 may include detecting an annotation trigger based on user input. The user input could be an audio input from the user speaking a specific word, a user touch on a trigger button or a graphical user interface, a gesture, or proximity. In one implementation, a microphone positioned on a physiological signal monitor can be used to listen for the annotation trigger when the user input is an audio input. The microphone can listen for the annotation trigger as previously described. The annotation trigger could be a predetermined sound. For example, when the user input is an audio input, the annotation trigger could be a predetermined sound such as a word or phrase and the word or phrase's synonyms. Particularly, the word or phrase that is the annotation trigger could be a wake word that the user has uttered. A wake word, also known as a trigger word or hotword, is a specific term or phrase programmed into a voice-activated device to initiate its listening mode. When a user utters the wake word, the device's speech recognition system becomes activated, prompting the device to listen attentively for further voice commands or interactions. This allows users to interact with the device hands-free, as the wake word serves as a cue for the device to start processing and interpreting subsequent spoken input.

In another implementation, when the user input is touch, the annotation trigger could be a trigger button that the user has touched. The trigger button could be a button that is specifically designated for the annotation trigger. When the user presses the button, the annotation trigger is activated. The trigger button could be a hard button, or a button displayed on a touch screen of a graphical user interface.

In another implementation, when the user input is a gesture, the annotation trigger could be the user making a specific hand motion. At least one sensor positioned on a physiological signal monitor can be used to monitor for the specific hand motion. For example, the hand motion could be a wave. Similarly, when the user input is proximity, the annotation trigger could be that the user is within a threshold distance from a physiological signal monitor. A sensor could be used to determine the user's distance.

In one example, the annotation trigger could be an event. For example, the user could provide input to a physiological monitor that annotation triggers should be based on particular events as a default. Once the user has set the default, the events may be the annotation trigger. The event could be the physiological signal monitor detecting that a device has been connected to the monitor, or an alarm on the physiological signal monitor.

The annotation trigger could be detected at any time. For example, the annotation trigger could be used to start a patient record. The annotation trigger would then be tied to the patient record, so that any subsequent annotation trigger would have an effect the patient record. The annotation triggers could also occur while streaming data at a physiological signal monitor.

Block 704 may include in response to detecting the annotation trigger, obtaining a first audio data segment containing speech uttered by a user. The first audio data segment could be obtained by a microphone, which may be the same microphone that detected the annotation trigger. The microphone may listen to and may record the user's speech in order to obtain the audio data segment. The content of the audio data segment could contain references to a list of predetermined annotations. Alternatively, the content of the audio data segment could be raw audio.

In one implementation the length of the first audio data segment is a predetermined duration. For example, the microphone may only listen for a predetermined duration, such as 30 seconds, one minute, or two minutes. Alternatively, the microphone may listen to the speech uttered by the user until the user is finished speaking. The duration of the first audio data segment may be determined by when the user has stopped, or discontinued, speaking.

One implementation of the method can include, in response to detecting the annotation trigger, activating at least one feature of a physiological signal monitor. Features of the physiological signal monitor could be actions that the physiological signal monitor is intended to carry out. For example, the physiological signal monitor could be a defibrillator. When the physiological signal monitor is a defibrillator, the annotation trigger could activate a feature of the defibrillator such as charge, or shock.

Block 706 may include determining that the first audio data segment containing speech includes at least one marker word that is associated with at least one marker event in a pre-defined set of marker events. The marker word could be determined to be present in the audio data segment using the previously mentioned speech recognition systems. The pre-defined set of marker events could be a list stored within the physiological signal monitor that includes a plurality of different events. The marker events could include a medical event, a note, a treatment, airway treatment, CPR, medication administration, patient response, user action, and/or patient record updates. Each event in the list could be associated with a marker word, and its synonyms. When the audio data segment contains the marker work, the marker event associated with it is also triggered. The marker event for a medical event could mean adding an indication that a medical event occurred at a specific time in a patient record or on patient data. The marker events for a note could mean adding a user's note to a patient record or on patient data. The medical event for a treatment could mean adding an indication that a specific patient treatment occurred at a specific time in a patient record or on patient data. The medical event for airway treatment could mean adding an indication that patient airway treatment occurred at a specific time in a patient record or on patient data. The medical event for CPR could mean adding an indication that CPR occurred at a specific time in a patient record or on patient data. The medical event for medication administration could mean adding an indication that a medication was administered to a patient at a specific time in a patient record or on patient data. The medical event for patient response could mean adding an indication that the patient responded to treatment in a specific way at a specific time in a patient record or on patient data. The medical event for user action could mean adding an indication that the user took an action at a specific time in a patient record or on patient data. The medical event for patient record updates could mean making changes to the data in the patient record.

In one implementation, in response to determining that the speech in the first audio data segment includes the at least one marker word that is associated with the at least one marker event, the method can include determining what the marker event includes. For example, the marker event can include a command to create the patient electronic health record. In response to determining that the marker event comprises creating the electronic health record, the method can include creating the electronic patient health record. Once the electronic patient health record is created, medical data, waveforms, notes, and annotations can be added to it. Alternatively, the marker event could be to annotate. In which case, annotations could be added to the electronic patient health record, including on data within the electronic patient health record.

Block 708 may include determining a marker time at which the annotation trigger was detected, or at which the first audio data segment containing speech was uttered. The marker time can be used to indicate a time at which some form of data, such as annotations and/or events, is added to the electronic patient health record. The physiological signal monitor that executes the method can include a clock in order to keep track of time. The marker time can be based on the time at which the physiological signal monitor detected the annotation trigger. The physiological signal monitor can imbed timestamp information into the metadata associated with the annotation. In addition to, or alternatively, the marker time can be based on the time at which the physiological signal monitor detected the first audio data segment containing speech. The physiological signal monitor can imbed timestamp information into the metadata associated with the first audio data segment, or the marker word that is associated with at least one marker event in a pre-defined set of marker events.

Block 710 may include annotating the patient electronic health record with the marker event that is associated with the at least one marker word, wherein the marker event is annotated within the patient electronic health record at a time point representing the marker time. For example, when the marker event is added to the patient electronic health record, it can include an indication of the time including the marker time that the marker event is associated with. In some implementations, the marker event could be annotated on a timeline. In other implementations, the marker event includes a note of the time.

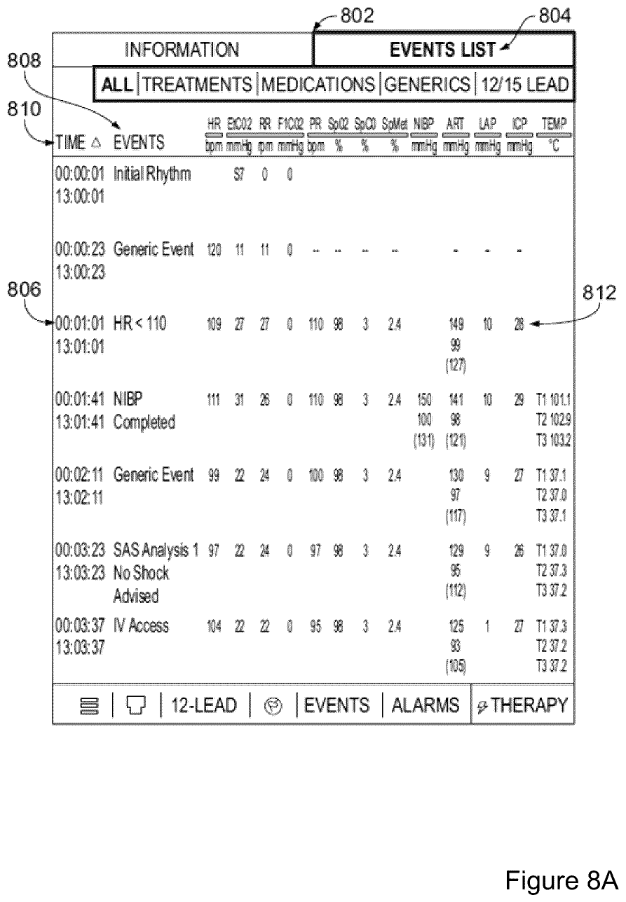

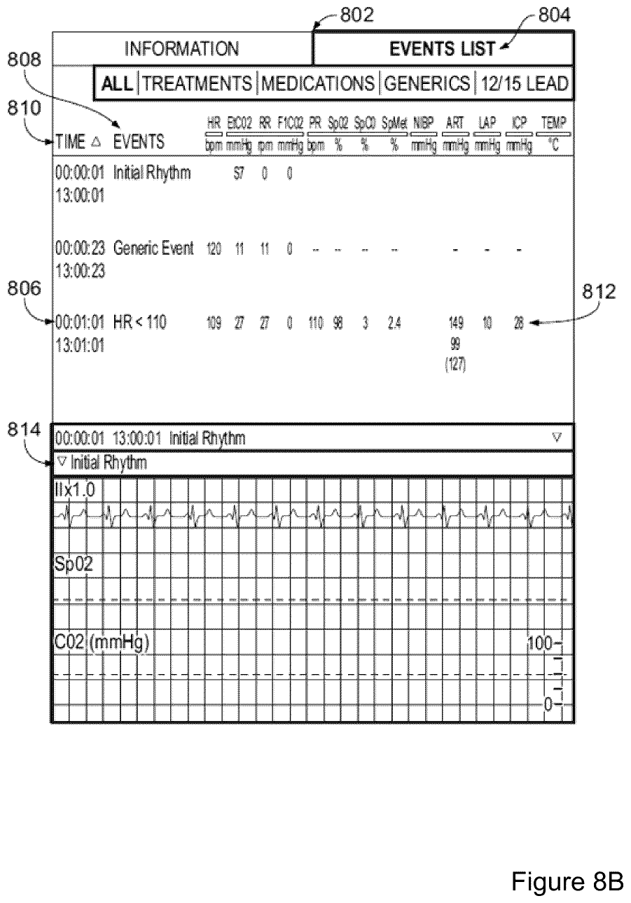

The marker event can be a note or indication of an event in a patient record. FIG. 8A illustrates a GUI 802 displaying a patient treatment event list 804 that has been added to the patient electronic health record. As illustrated, the patient treatment event list 804 includes at least one reference to an event 806 in the first real-time sensor data. Particularly, the GUI 802 can include a list of events 808 (i.e., an event list) and times 810 associated with the events 808. The list of events 808 may be sorted in ascending time order from when treatment begins. Each event may also include patient information 812. The patient information may include a plurality of physiological data associated with the event. For example, the list 808 includes an event 806 of HR<100. For the event 806, there is a time at which it happened and values for heart rate, EtCO2, non-invasive blood pressure, invasive blood pressure, and temperature at the time the event occurred. Each event on the patient treatment event list 804 may further include a link to a waveform 814 associated with the event 806.

FIG. 8B illustrates events that have been added to the patient electronic health record and include references to specific times in waveforms, which is data gathered by the physiological signal monitor. FIG. 8B illustrates the waveform 814 for the physiological data gathered using the sensors coupled to the external defibrillator may be displayed on GUI 802 for easy access with references to the events.

Some implementations of the method can include based on determining that the first audio data segment containing speech includes the at least one marker word that is associated with one of the pre-defined set of marker events, converting a portion of the first audio data segment following the at least one marker word to text and adding the text to a record of the marker event in the patient electronic health record. At least one of the marker events in the pre-defined set of marker events can be to transcribe the spoken words following the marker words and add the transcribed words to the marker event in the patient electronic health record. This can be so that dictated notes can be added to the patient electronic health record as written notes. That way, a user can add notes about a specific marker event while they are in the scene instead of needing to add notes at a later time.

Spoken words can be transcribed using speech recognition technology, which converts audio signals of spoken language into written text. This process can involve several steps: first, the audio input is captured by a microphone or recording device and converted into digital format. Next, a speech recognition system analyzes the audio waveform, segmenting it into phonetic units and identifying individual words and phrases. The system then matches these phonetic units to a pre-existing database of words and language models, using statistical algorithms and machine learning techniques to determine the most likely transcription for the spoken words. Finally, the transcribed text is outputted as written text, with punctuation and formatting applied to improve readability.

Some implementations of the method can also include determining that the text comprises an additional marker word that is associated with a second marker event of the pre-defined set of marker events. In other words, within the text converted form the spoken word there could be an additional marker word. The user could have used it during the first audio data segment to trigger an additional marker event. The method can include determining a second marker time at which the additional marker word was uttered in the same manner as previously described. The method could then include annotating the patient electronic health record with the second marker event that is associated with the additional marker word. The second marker event can be annotated within the patient electronic health record at a second time point representing the marker time. This could be used to matching the converted text to predetermined events, such as intubation.

Another implementation can include adding raw audio data to the patient electronic health record without transcribing it. The patient electronic health record can include a second audio data segment. The second audio segment can be a raw audio data segment that is added as an annotation within the patient electronic health record. The marker word within the first audio data segment could be the trigger that instructs the physiological signal monitor to add the second audio data segment to the patient electronic health record.

One implementation of the method can further include receiving a patient data stream generated from patient physiological data gathered by at least one physiological signal monitor. For example, the patient data stream could be ECG data. The method could include annotating the patient data stream with the marker event that is associated with the at least one marker word at the time point representing the marker time. The annotated patient data stream could be added to the patient electronic health record.

Block 712 may include adding the annotated patient electronic health record to a master medical record. The annotated data could be integrated into an existing electronic health record system used for maintaining a master record. This process can involve importing the annotated data, which may include additional notes, updates, or interpretations made by users, into the corresponding sections of the master record. The integration process attempts to make sure that the annotated data becomes seamlessly incorporated into the comprehensive patient record, preserving the chronological order of events and maintaining data integrity.

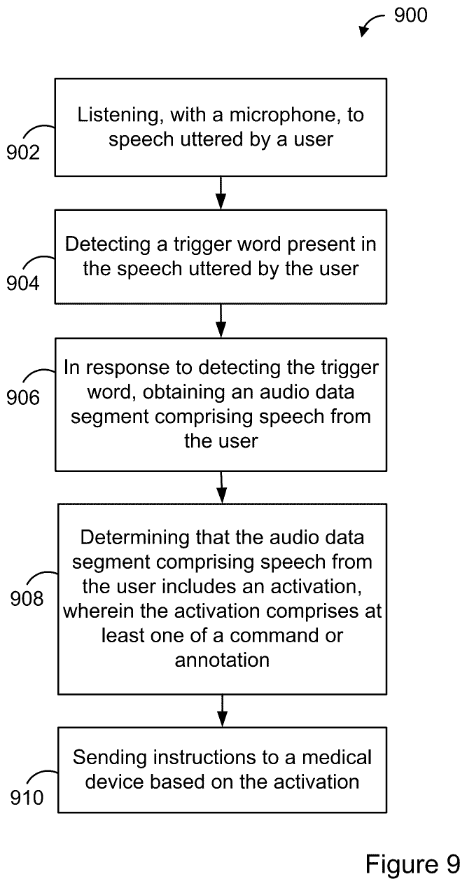

Those skilled in the art will understand that the flowchart of FIG. 9 herein illustrates functionality and operations of certain implementations of the present disclosure. In this regard, each block of the flowchart may represent a module, a segment, or a portion of program code, which includes one or more instructions executable by one or more processors for implementing specific logical functions or steps in the process. The program code may be stored on any type of computer readable medium, for example, such as a storage device including a disk or hard drive.

In addition, each block may represent circuitry that is wired to perform the specific logical functions in the process. Alternative implementations are included within the scope of the example implementations of the present application in which functions may be executed out of order from that shown or discussed, including substantially concurrent or in reverse order, depending on the functionality involved, as would be understood by those reasonably skilled in the art.

The method illustrated in FIG. 9, method 900, can include listening, with a microphone, to speech uttered by a user, detecting a trigger word present in the speech uttered by the user, in response to detecting the trigger word, obtaining an audio data segment comprising speech from the user, determining that the audio data segment comprising speech from the user includes an activation, wherein the activation comprises at least one of a command or annotation, and sending instructions to a physiological signal monitor based on the activation.

Block 902 can include listening, with a microphone, to speech uttered by a user. The microphone can listen to the speech uttered by the user in any of the ways previously described. For example, the microphone device 140 may be in a default passive listening mode. The microphone device 140 can capture acoustic waves produced by the user's vocal cords and surrounding environment. When a user speaks, their vocal cords vibrate, producing sound waves that propagate through the air. The microphone's diaphragm responds to these variations in air pressure, converting them into electrical signals. These signals, representing the speech waveform, are then transmitted to a processing system, which may include analog or digital signal processing algorithms designed to filter, enhance, and analyze the audio data.

Block 904 can include detecting a trigger word present in the speech uttered by the user. The trigger word can be detected in the speech uttered by the user in any of the ways previously described. For example, the trigger word could be a predetermined sound such as a word or phrase and the word or phrase's synonyms. Particularly, the word or phrase that is the trigger word could be a wake word that the user has uttered. A wake word, also known as a hotword, is a specific term or phrase programmed into a voice-activated device to initiate its listening mode. When a user utters the wake word, the device's speech recognition system becomes activated, prompting the device to listen attentively for further voice commands or interactions. This allows users to interact with the device hands-free, as the wake word serves as a cue for the device to start processing and interpreting subsequent spoken input.

Block 906 can include in response to detecting the trigger word, obtaining an audio data segment including speech from the user. The audio data segment including speech from the user can be obtained in any of the ways previously described. The audio data segment could be obtained by a microphone, which may be the same microphone that detected the trigger word. The microphone may listen to and may record a user's speech in order to obtain the audio data segment. The content of the audio data segment could contain references to a list of predetermined annotations. Alternatively, the content of the audio data segment could be raw audio.

In one implementation the length of the first audio data segment is a predetermined duration. For example, the microphone may only listen for a predetermined duration, such as 30 seconds, one minute, or two minutes. Alternatively, the microphone may listen to the speech uttered by the user until the user is finished speaking. The duration of the first audio data segment may be determined by when the user has stopped, or discontinued, speaking.

Block 908 can include determining that the audio data segment including speech from the user includes an activation, where the activation includes at least one of a command or annotation. Determining that the audio data segment including speech from the user includes an activation can be done in any of the ways previously described. For example, the activation could be a specific word or phrase and its synonyms. A speech recognition system could be used to determine that the activation is in the audio data segment. In one implementation, the activation is a command for a physiological signal monitor to perform an action. In another implementation, the activation is an instruction to make an annotation in a patient health record.

Block 910 can include sending instructions to a physiological signal monitor based on the activation. Once the activation is determined, the instructions can be sent based on the content of the activation. For example, the instructions could be to perform an action or to make an annotation on a patient record. One implementation of the method can include, activating at least one feature of a physiological signal monitor. Features of the physiological signal monitor could be actions that the physiological signal monitor is intended to carry out. For example, the physiological signal monitor could be a defibrillator. When the physiological signal monitor is a defibrillator, the annotation trigger could activate a feature of the defibrillator such as charge, or shock. Another implementation of the method can include annotating the patient record with notes from the user or markers for specific events.

The present disclosure also comprises the following clauses, with specific features laid out in dependent clauses, that may specifically be implemented as described in greater detail with reference to the configurations and drawings above.

Clause 1: A method for annotating a patient electronic health record comprising:
detecting an annotation trigger based on user input;
in response to detecting the annotation trigger, obtaining a first audio data segment containing speech uttered by a user;
determining that the first audio data segment containing speech comprises at least one marker word that is associated with at least one marker event in a pre-defined set of marker events;
determining a marker time at which the annotation trigger was detected, or at which the first audio data segment containing speech was uttered;
annotating the patient electronic health record with the marker event that is associated with the at least one marker word, wherein the marker event is annotated within the patient electronic health record at a time point representing the marker time; and
adding the annotated patient electronic health record to a master medical record.

Clause 2: The method of clause 1, further comprising:
receiving a patient data stream generated from patient physiological data gathered by at least one physiological signal monitor;
annotating the patient data stream with the marker event that is associated with the at least one marker word at the time point representing the marker time; and
adding the annotated patient data stream to the patient electronic health record.

Clause 3: The method of clause 1, further comprising:
in response to determining that the speech in the first audio data segment comprises the at least one marker word that is associated with the at least one marker event, determining that the marker event comprises a command to create the patient electronic health record; and in response to determining that the marker event comprises creating the electronic health record, creating the electronic patient health record.

Clause 4: The method of clause 1, wherein detecting the annotation trigger comprises detecting a predetermined sound with a microphone.

Clause 5: The method of clause 4, wherein detecting the annotation trigger comprises determining that the user has uttered a wake word.

Clause 6: The method of clause 1, wherein detecting the annotation trigger comprises determining that the user has pushed a trigger button.

Clause 7: The method of clause 1, wherein detecting the annotation trigger comprises detecting an event.

Clause 8: The method of clause 1, wherein the first audio data segment is a predetermined duration.

Clause 9: The method of clause 1, further comprising determining a duration of the first audio data segment by detecting when the speech uttered by the user has discontinued.

Clause 10: The method of clause 1, wherein the marker event comprises at least one of a medical event, a note, a treatment, or a medication.

Clause 11: The method of clause 1, further comprising:
based on determining that the first audio data segment containing speech comprises the at least one marker word that is associated with one of the pre-defined set of marker events, converting a portion of the first audio data segment following the at least one marker word to text and adding the text to a record of the marker event in the patient electronic health record.

Clause 12: The method of clause 11, further comprising:
determining that the text comprises an additional marker word that is associated with a second marker event of the pre-defined set of marker events;
determining a second marker time at which the additional marker word was uttered; and
annotating the patient electronic health record with the second marker event that is associated with the additional marker word, wherein the second marker event is annotated within the patient electronic health record at a second time point representing the marker time.

Clause 13: The method of clause 1, further comprising:
in response to detecting the annotation trigger, activating at least one feature of a physiological signal monitor.

Clause 14: The method of clause 1, wherein the patient electronic health record further comprises a second audio data segment, and wherein such second audio segment comprises a raw audio data segment that is annotated within the patient electronic health record using the marker word in the first audio data segment.

Clause 15: A system comprising:
a physiological signal monitor configured to monitor a patient;
a microphone device configured to obtain speech from a user associated with the physiological signal monitor;
a controller comprising at least one processor, at least one non-transitory data storage and a non-transitory computer-readable medium that stores a set of program instructions and a speech recognition system, wherein the at least one processor executes the program instructions stored in the at least one non-transitory data storage and executable by the at least one processor to carry out operations comprising:
   listening to speech uttered by a user;
   detecting an action trigger present in the speech uttered by the user;
   in response to detecting the action trigger, obtaining an audio data segment comprising speech from the user;
   determining that the audio data segment comprising speech from the user includes a physiological signal monitor command;
   determining if the physiological signal monitor command is valid; and
   instructing the physiological signal monitor to perform a physiological signal monitor action associated with the physiological signal monitor command.

Clause 16: The system of clause 15, wherein determining if the physiological signal monitor command is valid comprises determining any false interpretation in the audio data segment.

Clause 17: The system of clause 15, wherein determining if the physiological signal monitor command is valid comprises requiring and obtaining voice confirmation of the physiological signal monitor command.

Clause 18: The system of clause 15, further comprising:
determining that the physiological signal monitor command is invalid; and
in response to determining that the physiological signal monitor command is invalid, requesting a further command from the user.

Clause 19: The system of clause 15, further comprising:
determining that the physiological signal monitor command is valid; and
in response to determining that the physiological signal monitor command is valid, requesting a confirmation command from the user, wherein the confirmation command comprises at least one of a voice confirmation or a touch confirmation.

Clause 20: The system of clause 15, wherein the physiological signal monitor is a defibrillator.

Clause 21: The system of clause 20, wherein the physiological signal monitor command is at least one of charge, shock, annotate, or display.

Clause 22: A method for physiological signal monitor control comprising:
listening, with a microphone, to speech uttered by a user;
detecting a trigger word present in the speech uttered by the user;
in response to detecting the trigger word, obtaining an audio data segment comprising speech from the user;
determining that the audio data segment comprising speech from the user includes an activation, wherein the activation comprises at least one of a command or annotation; and
sending instructions to a physiological signal monitor based on the activation.

The foregoing summary is illustrative only and is not intended to be in any way limiting. In addition to the illustrative aspects, implementations, and features described above, further aspects, implementations, and features will become apparent by reference to the figures and the following detailed description.

What is claimed is:

1. A method for annotating a patient electronic health record comprising:
detecting an annotation trigger based on user input;
in response to detecting the annotation trigger, obtaining a first audio data segment containing speech uttered by a user;
determining that the first audio data segment containing speech comprises at least one marker word that is associated with at least one marker event in a pre-defined set of marker events;
determining a marker time at which the annotation trigger was detected, or at which the first audio data segment containing speech was uttered;
annotating the patient electronic health record with the marker event that is associated with the at least one marker word, wherein the marker event is annotated within the patient electronic health record at a time point representing the marker time; and
adding the annotated patient electronic health record to a master medical record.

2. The method of claim 1, further comprising:
receiving a patient data stream generated from patient physiological data gathered by at least one physiological signal monitor;
annotating the patient data stream with the marker event that is associated with the at least one marker word at the time point representing the marker time; and
adding the annotated patient data stream to the patient electronic health record.

3. The method of claim 1, further comprising:
in response to determining that the speech in the first audio data segment comprises the at least one marker word that is associated with the at least one marker event, determining that the marker event comprises a command to create the patient electronic health record; and in response to determining that the marker event comprises creating the patient electronic health record, creating the electronic patient health record.

4. The method of claim 1, wherein detecting the annotation trigger comprises detecting a predetermined sound with a microphone.

5. The method of claim 4, wherein detecting the annotation trigger comprises determining that the user has uttered a wake word.

6. The method of claim 1, wherein detecting the annotation trigger comprises determining that the user has pushed a trigger button.

7. The method of claim 1, wherein detecting the annotation trigger comprises detecting an event.

8. The method of claim 1, wherein the first audio data segment is a predetermined duration.

9. The method of claim 1, further comprising determining a duration of the first audio data segment by detecting when the speech uttered by the user has discontinued.

10. The method of claim 1, wherein the marker event comprises at least one of a medical event, a note, a treatment, or a medication.

11. The method of claim 1, further comprising:
based on determining that the first audio data segment containing speech comprises the at least one marker word that is associated with one of the pre-defined set of marker events, converting a portion of the first audio data segment following the at least one marker word to text and adding the text to a record of the marker event in the patient electronic health record.

12. The method of claim 11, further comprising:
determining that the text comprises an additional marker word that is associated with a second marker event of the pre-defined set of marker events;
determining a second marker time at which the additional marker word was uttered; and
annotating the patient electronic health record with the second marker event that is associated with the additional marker word, wherein the second marker event is annotated within the patient electronic health record at a second time point representing the marker time.

13. The method of claim 1, further comprising:
in response to detecting the annotation trigger, activating at least one feature of a physiological signal monitor.

14. The method of claim 1, wherein the patient electronic health record further comprises a second audio data segment, and wherein such second audio segment comprises a raw audio data segment that is annotated within the patient electronic health record using the marker word in the first audio data segment.

15. A system comprising:
a physiological signal monitor configured to monitor a patient;
a microphone device configured to obtain speech from a user associated with the physiological signal monitor;
a controller comprising at least one processor, at least one non-transitory data storage and a non-transitory computer-readable medium that stores a set of program instructions and a speech recognition system, wherein the at least one processor executes the program instructions stored in the at least one non-transitory data storage and executable by the at least one processor to carry out operations comprising:
listening to speech uttered by a user;
detecting an action trigger present in the speech uttered by the user;
in response to detecting the action trigger, obtaining an audio data segment comprising speech from the user;
determining that the audio data segment comprising speech from the user includes a physiological signal monitor command;
determining if the physiological signal monitor command is valid; and
instructing the physiological signal monitor to perform a physiological signal monitor action associated with the physiological signal monitor command.

16. The system of claim 15, wherein determining if the physiological signal monitor command is valid comprises determining any false interpretation in the audio data segment.

17. The system of claim 15, wherein determining if the physiological signal monitor command is valid comprises requiring and obtaining voice confirmation of the physiological signal monitor command.

18. The system of claim 15, further comprising:
determining that the physiological signal monitor command is invalid; and
in response to determining that the physiological signal monitor command is invalid, requesting a further command from the user.

19. The system of claim 15, further comprising:
determining that the physiological signal monitor command is valid; and
in response to determining that the physiological signal monitor command is valid, requesting a confirmation command from the user, wherein the confirmation command comprises at least one of a voice confirmation or a touch confirmation.

20. The system of claim 15, wherein the physiological signal monitor is a defibrillator.

21. The system of claim 20, wherein the physiological signal monitor command is at least one of charge, shock, annotate, or display.

22. A method for physiological signal monitor control comprising:
listening, with a microphone, to speech uttered by a user;
detecting a trigger word present in the speech uttered by the user;
in response to detecting the trigger word, obtaining an audio data segment comprising speech from the user;
determining that the audio data segment comprising speech from the user includes an activation, wherein the activation comprises at least one of a command or annotation; and
sending instructions to a physiological signal monitor based on the activation.
